(19) United States
(12) Patent Application Publication
(10) Pub. No.: US 2006/0272648 A1
(43) Pub. Date: Dec. 7, 2006

(54) Sex aid incorporating membrane edges

(76) Inventor: Jeffrey James Anderson, Orlando, FL (US)

Correspondence Address: JEFFREY ANDERSON, PO BOX 590262, ORLANDO, FL, 32859, US

(21) Appl. No.: 11/143,048

(22) Filed: Jun. 2, 2005

(51) Int. Cl. A61F 6/02

(52) U.S. Cl. 128/842

(57) ABSTRACT

A sexual aid that provides ultra realistic sensation of skin contact by utilizing one or more edges of a thin membrane as the primary contact with the skin of the user. Both a simulated male genitalia and a simulated female genitalia version of the invention is described here. The simulated female genitalia is chiefly comprised of a tubular rubber membrane stretched over a support ring to form two flat stretched circular membranes each with a stretchable center hole. The simulated male genitalia is chiefly comprised of multiple circular discs mounted at their center and sequentially along a central support rod. Each disc is tapered to transition to a thin circular membrane edge at its circumference. In both gender versions, primary contact with the user is with the separate membrane edges that create a unique inward or outward pressure against the user that is operationally separated from the other membrane edges.

### FIELD OF THE INVENTION

The present invention relates to sexual aids or sex therapy devices and, more particularly, to a sex device that incorporates one or more edges of an elastic membrane as the primary contact point to very accurately simulate the sensation of a sex partner.

### BACKGROUND OF THE INVENTION

Sex aids have the potential for providing sexual gratification and sexual therapy for thousands of users both in and out of marriage and relationships. Sexual gratification within relationships is a chief reason for dissolution of relationships and also the biggest reason partners become unfaithful and possibly expose themselves to sexually transmitted disease. If a more realistic sex aid were developed, sexual fulfillment could be experienced among partners, even when one partner is sexually dysfunctional, inexperienced, or in need of more self control during sex.

Sex aids in the past have varied wildly in all aspects except for their attention given to the delicate and hard to replicate sensation of a partner's actual skin. In spite of all the creative devises to date, they have typically relied on a continuous single surface the rides flatly against the genitalia. Other than minute bumps or ridges on this surface, the only significant sensation of starting or stopping contact with this surface is the complete entry or separation with the device.

These previous solutions provide a very poor simulation at best.

It is therefore an object of the invention to provide a sex aid for a male user with vastly improved tactile simulation of a female genitalia.

It is another object of the invention to provide a sex aid for a female user with vastly improved tactile simulation of a male genitalia.

### SUMMARY OF THE INVENTION

In accordance with the present invention, there is provided a sexual aid that provides ultra realistic sensation of skin contact by utilizing one or more edges of a thin elastic membrane as the primary contact with the skin of the user. Both a simulated male genitalia and a simulated female genitalia version of the invention is described here.

The simulated female genitalia is chiefly comprised of a three inch section of a thin rubber tubular membrane stretched over a ring about three inches in diameter. This rubber membrane is draped over both sides of the ring to form two flat stretched circular membranes. Each resulting circular membrane has a resultant opening hole hole in its center of about a half inch in diameter to allow entry of the male user's genitalia. The ring used also has a width of about three quarter inch to hold both membranes in parallel with each other and three quarter inch apart. More of these membranes can be stacked for increased sensation. The primary contact with the user is with the membrane edges. The various membrane edges can therefore be located at different locations with different diameters, each creating a separate and unique outward pressure against the user that is operationally separated from the other membrane edges.

The simulated male genitalia is chiefly comprised of a support rod provided as a mounting point for multiple membrane discs that are fabricated with a unique taper radially from the center resulting in a very soft thin membrane edge on the circumference of each disc. Multiple discs can be used to further replicate sensation and general shape of male genitalia and provide a very soft outward pressure experience by the user that is perceived by the user as soft natural skin. The primary contact with the user is with the membrane edges. The various membrane edges can therefore be located at different locations with different diameters, each creating a separate and unique outward pressure against the user that is operationally separated from the other membrane edges.

### BRIEF DESCRIPTION OF THE DRAWINGS

A complete understanding of the present invention may be obtained by reference to the accompanying drawings, when considered in conjunction with the subsequent, detailed description, in which:

FIG. 1 is a perspective view of a simulated female version of invention; and

FIG. 2 is a side view of a simulated male version of invention.

For purposes of clarity and brevity, like elements and components will bear the same designations and numbering throughout the FIGURES.

### DESCRIPTION OF THE PREFERRED EMBODIMENT

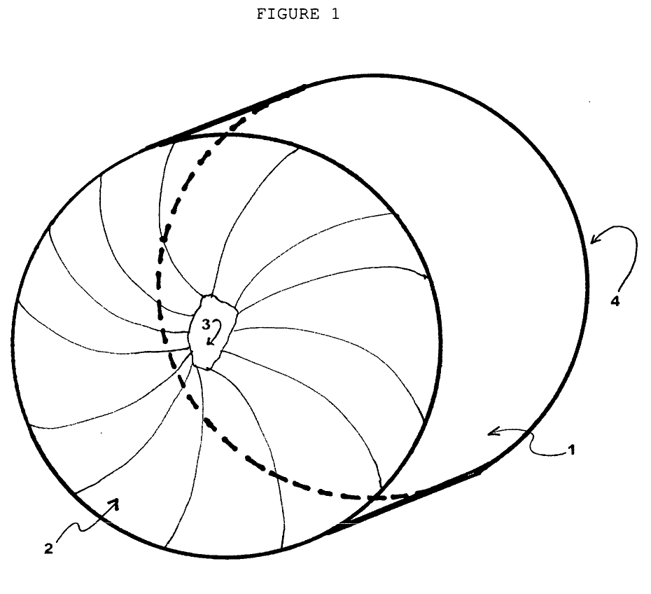

FIG. 1 is a perspective view of a version of the invention that simulates female genitalia. A stretched elastic circular membrane 2 that is tubular in shape and about three inches in length and one inch in diameter, is stretched over a Support ring 1 larger in diameter than the membranes prestretched diameter. The ends of the membrane now reside on opposing sides of the ring and stretch inward toward the resultant opening at membrane edge 3 and yield two holes that are each somewhat smaller than a typical penis. The front side hole and the additional membrane on backside of ring (not shown) 4 with it's additional membrane edge provided via element 4 (not shown) 5 are concentrically in line with each other and are spaced about ¾ inch apart to separate and replicate this sensation along the penis. Additional rings can be housed or positioned in line with one another to further replicate and elongate the sensation. The primary contact with the user is with the membrane edges. Contact with the user between the membranes is relatively non existent.

In operation, a male user of the simulated female genitalia shown in FIG. 1 would lubricate the membrane edges and then insert his penis through some or all of the membrane holes. The membrane edges would flow inward and outward as the user moves inward and outward with life-like pressures continuous maintained.

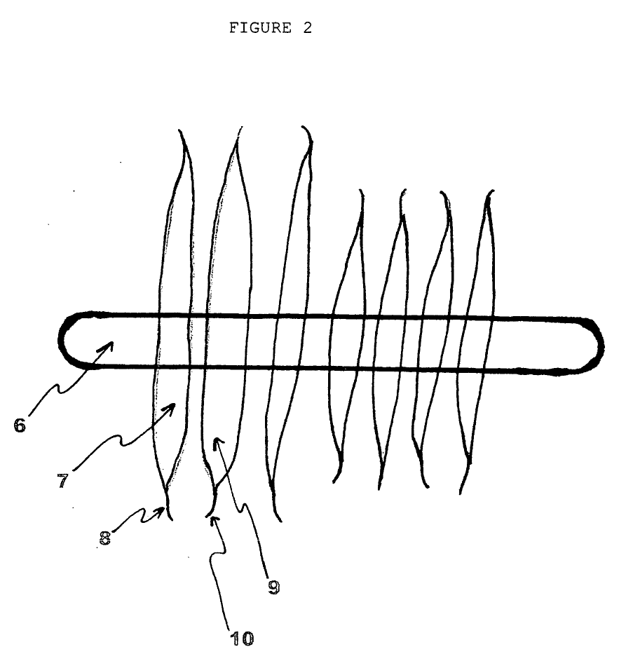

FIG. 2 is a perspective view of a version of the invention that simulates male genitalia. Firstly, a support rod 6 is provided as a mounting point for the membrane discs. Secondly, a tapered circular membrane disc 7 is fabricated with a unique taper radially from the center. Moving outward from the discs center, the disc are stiff and wide to support mounting along the support rod 6. At the discs circumference, the taper occurs much more rapidly resulting in a very soft thin membrane edge of the first membrane disc 8. An additional membrane disc 9 with it's additional membrane edge provided via element 9 (not shown) 10 is mounted along the support rod 6 to further replicate the general shape of male genitalia. This results in a very soft outward pressure experience by the user mainly at the periphery of the overall devise and is perceived by the user as soft natural skin. Multiple discs can be used to further replicate and elongate the sensation. The primary contact with the user is with the membrane edges. Contact with the user between the membranes is relatively non existent.

In operation, a female user of the simulated male genitalia shown in FIG. 2 would lubricate the membrane edges and then insert the entire device partially or fully into her vagina. The membrane edges would flow inward and outward as the user moves the entire device inward and outward with life-like pressures continuous maintained.

As an additional element of realism and to enclose, reveal, conceal, and protect the the male embodiment of the invention, a cloth Outer male garment 12 optionally including zippers and buttons can optionally be used that is representative of clothes typically associated with a common male.

As an additional element of realism and to enclose, reveal, conceal, and protect the the female embodiment of the invention, a cloth Outer female garment 11 optionally including zippers and buttons can optionally be used that is representative of clothes typically associated with a common female.

Since other modifications and changes varied to fit particular operating requirements and environments will be apparent to those skilled in the art, the invention is not considered limited to the example chosen for purposes of disclosure, and covers all changes and modifications which do not constitute departures from the true spirit and scope of this invention.

Having thus described the invention, what is desired to be protected by Letters Patent is presented in the subsequently appended claims.

What is claimed is:

1. A sex aid incorporating membrane edges for providing ultra realistic sexual sensation comprising:
a support ring, for positioning and holding the outwardly streched membrane in a close proximity but not touching the users penis and also holding the second membrane on the backside of the ring at a fixed but seperated distance from the frontside membrane;
a stretched elastic circular membrane, for guiding the users penis and providing a mild tension against and encircling the users penis, peripherally supported to said Support ring;
a resultant opening at membrane edge, for providing the actual contact area and sensation for the user, adjacently delineated to said stretched elastic circular membrane;
a support rod, for providing the basic support for the multiple circular membrane discs;
a tapered circular membrane disc, for outwardly transistioning support from the central support to the thin delicate mebrane at the discs circular perimiter, rigidly connected to said support rod;
a thin membrane edge of the first membrane disc, for presenting a soft contact surface to the user, peripherally molded to said tapered circular membrane disc;
an outer female garment, for providing additional realism and enclosure for the device, peripherally fitted to said stretched elastic circular membrane; and
an outer male garment, for providing additional realism and enclosure for the device, peripherally fitted to said tapered circular membrane disc.

2. A sex aid incorporating membrane edges for providing ultra realistic sexual sensation comprising:
a support ring, for positioning and holding the outwardly streched membrane in a close proximity but not touching the users penis and also holding the second membrane on the backside of the ring at a fixed but seperated distance from the frontside membrane;
a stretched elastic circular membrane, for guiding the users penis and providing a mild tension against and encircling the users penis, peripherally supported to said Support ring;
a resultant opening at membrane edge, for providing the actual contact area and sensation for the user, adjacently delineated to said stretched elastic circular membrane;
an additional membrane on backside of ring (not shown), for providing with optionally different location and diameter, support for an additional membrane edge thus creating seperate and unique inward pressure against the user,
an additional membrane edge provided via element 4 (not shown), for providing another soft contact surface to the user that is operationally seperated from the other membrane edges, operationally separated to said stretched elastic circular membrane;
a support rod, for providing the basic support for the multiple circular membrane discs;
a tapered circular membrane disc, for outwardly transistioning support from the central support to the thin delicate mebrane at the discs circular perimiter, rigidly connected to said support rod;
a thin membrane edge of the first membrane disc, for presenting a soft contact surface to the user, peripherally molded to said tapered circular membrane disc;
an additional membrane disc, for providing with optionally different location and diameter, support for an additional membrane edge thus creating seperate and unique outward pressure against the user,
rigidly connected to said support rod;
an additional membrane edge provided via element 9 (not shown), for providing another soft contact surface to the user that is operationally seperated from the other membrane discs
peripherally molded to said additional membrane disc, and operationally separated to said thin membrane edge of the first membrane disc;
an outer female garment, for providing additional realism and enclosure for the device, peripherally fitted to said stretched elastic circular membrane; and
an outer male garment, for providing additional realism and enclosure for the device, peripherally fitted to said tapered circular membrane disc.
